(19) United States
(12) Patent Application Publication
(10) Pub. No.: US 2012/0296172 A1
(43) Pub. Date: Nov. 22, 2012

(54) SURGICAL RETRACTOR APPARATUS AND METHOD

(76) Inventors: Raymond B. Raven, III, Pasadena, CA (US); Dan A. Zlotolow, Wyndmoor, PA (US)

(21) Appl. No.: 13/112,853

(22) Filed: May 20, 2011

(51) Int. Cl. A61B 1/32

(52) U.S. Cl. 600/231

(57) ABSTRACT

A surgical retractor is provided to retain tissue in a retracted position and expose an interior surface during a surgical procedure. The surgical retractor is self-retaining and includes opposing blades that are removably mountable on arms of the surgical retractor via a ball-and-socket assembly that permits swiveling and hinging of the opposing blades independent from each other and the arms. Each of the opposing blades have a plurality of talons with a curved surface to slidably receive and partially enwrap bone matter so that the plurality of talons securely engage the bone matter while permitting slidable adjustment therebetween.

### CROSS-REFERENCE TO RELATED APPLICATION

This patent application claims priority to U.S. patent application Ser. No. 29/376,374 filed Oct. 6, 2010, the entire contents of which is herein incorporated by reference in its entirety.

### BACKGROUND OF THE INVENTION

1. Field of the Invention

The present general inventive concept relates generally to medical instruments, and more particularly, to a surgical retractor to retain tissue in a retracted position and expose an interior surface during a surgical procedure.

2. Description of the Related Art

A retractor is a surgical instrument that allows a surgeon to separate edges of a surgical incision or wound. Conventional retractors generally encompass a handheld tool having a narrow blade that can be used to separate tissue and hold the tissue in a retracted state so that a surgical site is exposed. The narrow blade is typically affixed to a single arm or a narrow blade on a pair of linked pivoting arms.

Conventional retractors generally require a surgeon to maintain the tissue in the retracted state substantially throughout an entire surgical procedure. During the course of the surgical procedure, it is likely that the blade of the retractor will lose its grip on the tissue (e.g., due to the slippery nature of tissue), and that the inadvertent movement of the retractor (e.g., due to movement of the patient or the surgeon) will cause the retractor to deviate from the surgical site. Additionally, during the course of the surgical procedure, it is possible that the surgeon may desire a different perspective of the surgical site.

Such slips and movement, whether inadvertent or advertent, lengthen the time period required of the surgical procedure, which increases trauma to the patient thus extending recover time of the patient from the surgical procedure, elevates the risks associated with the surgical procedure, and increases the costs of the surgical procedure.

One such conventional retractor-type device is disclosed in U.S. Pat. No. 5,931,777 to Sava, the entire contents of which is herein incorporated by reference in its entirety. Sava provides a complex device that penetrates bone matter of a patient in order to maintain its position in the patient once established by a surgeon which is undesirable for two reasons. First, it is undesirable to make an incision or otherwise damage any part of the patient via penetrating or the like as such necessarily increases trauma and recovery time. In fact, intentional damage to a patient caused by a surgeon is typically only done only out of necessity. Second, very few surgeries include bone matter adjacent to a surgical field that would permit use of the Sava device as disclosed. In fact, the Sava device is only illustrated in use during spinal surgery.

Thus, there is a need for a retractor that does not suffer from the limitations of conventional retractors, is versatile to permit use in a wide variety of applications, has a simple design that is easy to use, and does not prolong recovery time or expenses of the patient.

### SUMMARY OF THE INVENTION

A principal object of the present general inventive concept is to provide a retractor that remedies the aforementioned deficiencies in conventional retractors and is ideal for retracting tissue during treatment of any tubular bone fracture including but not limited to the radius, ulna, femur, humerus, fibula, and clavicle.

Another object of the general inventive concept is to provide a retractor having a plurality of blades that are operable to retain tissue at a surgical site of a patient in a retracted position by leveraging on a portion of the patient at or adjacent to the surgical site so that the surgical site is exposed throughout the course of a surgical procedure. The surgical retractor is self-retaining and includes the plurality of blades are removably mountable on arms of the surgical retractor via a ball-and-socket assembly that permits swiveling and hinging of the opposing blades independent from each other and the arms, i.e., movement of each blade relative to the arms along up to three axes, e.g., an X, Y, and/or Z axes, or along up to three different planes.

Another object of the general inventive concept is to provide a retractor that is operable to slidably lever on a portion of a surgical site (e.g., a bone) during movement of the retractor from a first configuration (e.g., a stored configuration), to a second configuration (e.g., an in-use configuration) so that tissue is retracted from the surgical site and the surgical site is maximally exposed and subsequent adjustment of the retractor is facilitated via the slidable levering of the retractor.

Another object of the general inventive concept is to provide a retractor having a blade with edge portions and a talon that cooperatively form a cavity at a center of the blade.

Another object of the general inventive concept is to provide a retractor for use at a surgical site. The retractor has a tissue-contact portion and a surgical-site contact portion. The tissue-contact portion is of a first size and shape to maximize contact of the retractor with the tissue while conforming to an opening at the surgical site. The surgical-site contact portion is of a second size and shape to minimize contact of the retractor with the surgical site.

Another object of the general inventive concept is to provide a retractor having a universal coupler that is sized and shaped to connect with blades of different shapes and/or sizes to enable the retractor to adapt to various applications of the retractor. For instance, the present invention is operable to permit substitution of a first blade with a second blade of relatively longer length to enable use of the retractor in applications requiring deeper insertion into areas with thicker and/or deeper soft tissue.

Another object of the general inventive concept is to provide a retractor and method of use that is easy to use, comparatively simple to manufacture, and especially well adapted for the intended usage thereof.

The aforementioned objects and advantages of the present general inventive concept may be achieved by providing a self-retaining retractor assembly including an elongated body having opposing arms that are hinged together at a connection point that is spaced from either end of the elongated body. The spacer may be coupled to and depend from each of the arms. Each of the spacers may have a plurality of axes and be operable to independently pivot with respect to the elongated body about each of the plurality of axes. The spacer may be operable to partially enwrap a portion of a surgical site.

The self-retaining retractor assembly may further include a coupling assembly operable to permit independent removal of one or both spacers from the arms. The coupling assembly may be a ball-and-socket coupling assembly having a ball depending from each of the arms that may be operable to be received via a snap-fit into an aperture in each of the spacers. The aperture may be defined by a circumferential wall having a degree of resiliency. The coupling assembly may provide each of the spacer with a first degree of pivot about the X and Z axes, and a second degree of pivot about the Y axis. The first degree of pivot may be defined by an annular ring on each of the arms. Each of the annular rings may limit the first degree of pivot of each of the spacers to about 45 degrees. The second degree of pivot may be perpetual or 360 degrees. The plurality of axes of each the spacers may intersect at the coupling assembly of each of the spacers. Each of the spacers may include a face surface with side edge portions and a bottom edge portion. The bottom edge portion may have at least one tooth or talon depending therefrom, or may have two, three or more talons of equal size and/or shape, or different size and/or shape depending therefrom. The side edge portion and the at least one talon may be concave to form a cavity on each of the face surfaces.

The spacer may include at least one talon having a curved surface that may be operable to partially enwrap the portion of the surgical site. The spacer may include two talons each having a curved surface that each may be operable to cooperatively pivot and/or align the spacer with respect to the elongated body when the curved surfaces abut the portion of the surgical site. The talons may be slidable along the portion of the surgical site anytime during use of the self-retaining retractor. The self-retaining retractor does not penetrate any part of a patient (e.g., bone matter). The spacer may include two parallel curved talons with a curved wall extending perpendicular from either side of the two talons to collectively form and surround a cavity between the talons and walls.

The self-retaining retractor assembly may further include a gripping element on one end of the elongated body having a plurality of finger holes. The self-retaining retractor assembly may further include a locking mechanism on the body that is operable to lock the arms in one of a plurality of orientations with respect to each other. The self-retaining retractor assembly may further include a spring on the locking mechanism that is operable to bias the locking mechanism to a locked configuration. The spacers may be radiolucent blades. The connection point may be a hinge that is operable to permit movement of at least one of the arms with respect to the other of the arms.

The aforementioned objects and advantages of the present general inventive concept may further be achieved by providing a surgical retractor including a plurality of arms that may be hinged together, and a blade coupled to each of the arms via a friction-fit engagement. The present general inventive concept may include a divider between each of the plurality of arms and each of the blades that may be operable to define a limited range of movement the blade and the arm, and/or separate or at least facilitate separation of the blade from the arm if the blade exceeds the limited range of movement in any one of a plurality of directions. The limited range of movement provided to each of the blades may include a plurality of axes. The plurality of axes may include any one or combination of an X axis, a Y axis, and a Z axis. The plurality of axes may have an intersection point on the blade.

The aforementioned objects and advantages of the present general inventive concept may further be achieved by providing a method of surgery including the steps of providing a retractor having a plurality of opposing blades depending from hinged arms that are operable to selectively define a void between the plurality of blades, setting the hinged arms in a first configuration with respect to each other, the first configuration defining a first plurality of axes of each of the plurality of blades and permitting independent movement of the plurality of blades along the first plurality of axes, inserting the plurality of blades at least partially into a surgical area with the hinged arms in the first configuration, partially enwrapping a portion of the surgical site via the spacer, and retracting an object or tissue adjacent to the surgical area by moving the hinged arms from the first configuration to a second configuration.

The second configuration may define a second plurality of axes of each of the plurality of blades and permit independent movement of the plurality of blades along the second plurality of axes. The first and second plurality of axes may each include an X axis, a Y axis, and a Z axis. The X, Y and Z axes may share a common intersection point on the blade.

The blades may be removable from the arms and may be a set of blades selected from a plurality of blades having different shapes and sizes, and the set of blades may be of equal size and/or shape or different size and/or shape.

The method may further include the step of pivoting the spacer with respect to the elongated body when the portion of the spacer partially abuts and/or enwraps the portion of the surgical site. The portion of the spacer may be one talon or two talons that may have a curved surface to receive the portion of the surgical site. The curved surface may be contoured with respect to the portion of the surgical site. The portion of the surgical site may be a bone.

The method may further include the step of levering a portion of the blades on a portion of the surgical area as the hinged arms are moved from the first configuration to the second configuration. The method may further include the step of slidable moving the portion of the blades along the portion of the surgical site after the hinged arms are moved to the second configuration.

The foregoing and other objects are intended to be illustrative of the present general inventive concept and are not meant in a limiting sense. Many possible embodiments of the present general inventive concept may be made and will be readily evident upon a study of the following specification and accompanying drawings comprising a part thereof. Various features and subcombinations of present general inventive concept may be employed without reference to other features and subcombinations. Other objects and advantages of this present general inventive concept will become apparent from the following description taken in connection with the accompanying drawings, wherein is set forth by way of illustration and example, an embodiment of this present general inventive concept and various features thereof.

### BRIEF DESCRIPTION OF THE DRAWINGS

A preferred embodiment of the invention, illustrative of the best mode in which the applicant has contemplated applying the principles, is set forth in the following description and is shown in the drawings.

FIG. 1 is a perspective view of a retractor in accordance with an exemplary embodiment of the present inventive concept, illustrating the retractor having a plurality of blades connected to arms of the retractor;

FIG. 2 is a top plan view of the retractor illustrated in FIG. 1;

FIG. 3 is an elevated side view of the retractor illustrated in FIG. 1;

FIG. 4 is a magnified perspective view of the blades and the arms of the retractor illustrated in FIG. 1 with one of the blades exploded from one of the arms;

FIG. 5 is an elevated rear view of one of the blades illustrated in FIG. 1;

FIG. 6 is an elevated side view of one of the blades illustrated in FIG. 1;

FIG. 7 is a top-plan view of one of the blades illustrated in FIG. 1;

FIG. 8 is a perspective view of the retractor illustrated in FIG. 1, illustrating the retractor in use with the blades levered against a bone in a first configuration; and

FIG. 9 is a perspective view of the retractor illustrated in FIG. 1, illustrating the retractor in use with the blades levered against a bone in a second configuration.

The drawing figures do not limit the present inventive concept to the specific embodiments disclosed and described herein. The drawings are not necessarily to scale, emphasis instead being placed upon clearly illustrating the principles of the illustrated embodiment.

### DETAILED DESCRIPTION OF THE PREFERRED EMBODIMENTS

The present inventive concept is susceptible of embodiment in many forms. While the drawings illustrate, and the specification describes, certain embodiments of the invention, it is to be understood that such disclosure is by way of example only. The principles of the present inventive concept are not limited to the particular disclosed embodiments.

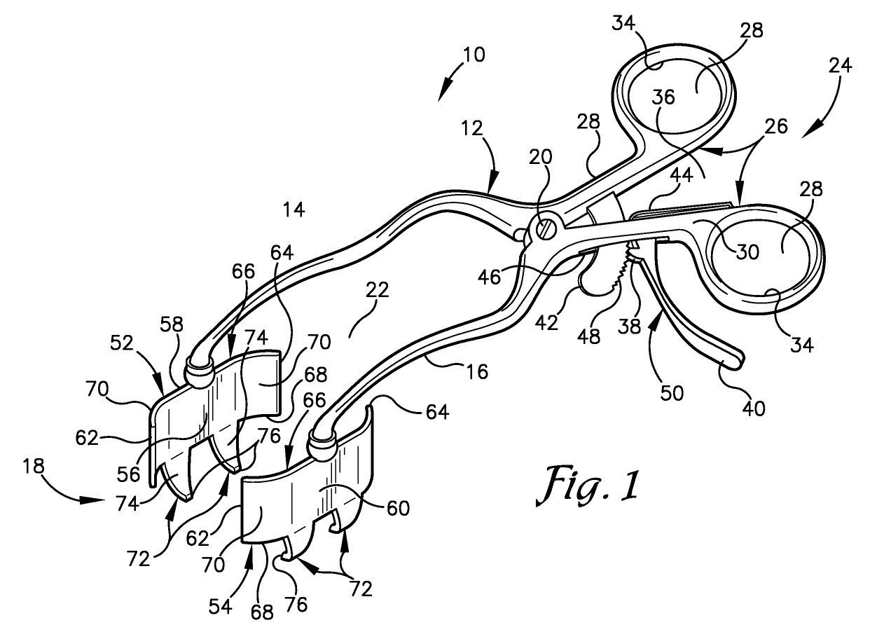

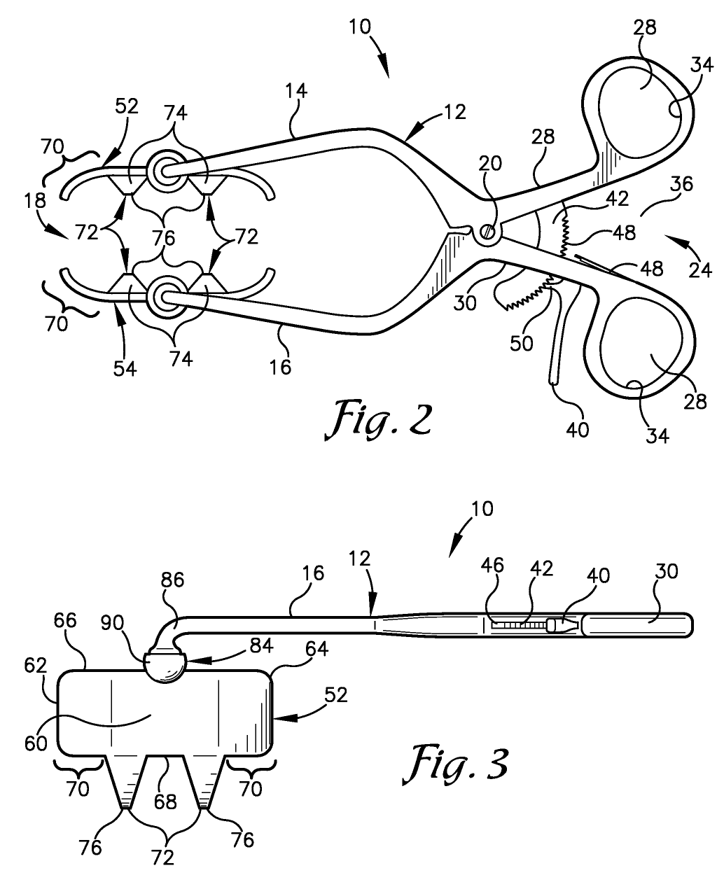

With initial reference to FIG. 1, a surgical retractor 10 in accordance with the exemplary embodiment of the present inventive concept is illustrated. The retractor 10 has an elongated body 12 that is formed from stainless steel or like material. The elongated body 12 includes opposing arms 14, 16 at a distal end 18 thereof. The opposing arms 14, 16, that are pivotably secured at a connection point or hinge 20 on the elongated body 12. The hinge 18 is spaced from either end of the elongated body 12 and is operable to permit movement of one or both of the opposing arms 14, 16 relative to one another. In this manner, the opposing arms 14, 16 may be selectively distanced from each other in various configurations so that a void 22 between the opposing arms 14, 16 is thereby defined. A size of the void 22 is variably and selectively determined by the configuration of the opposing arms 14, 16.

On a side of the hinge 20 opposite to the opposing arms 14, 16, i.e., at a proximal end 24 of the elongated body 12, is a grip assembly 26. The grip assembly 26 includes opposing extensions 28, 30 that are respectively connected to opposing arms 14, 16. Each of the opposing extensions 28, 30 include an aperture 32 defined by a generally circumferential edge 34 of each of the opposing extensions 28, 30. Each aperture 32 is sized and shaped to receive finger(s) and/or thumb(s) of a user or surgeon therethrough to facilitate use and manipulation of the retractor 10 by the surgeon. For purposes herein, the distal end 18 of the retractor 10 or any part thereof is the end closest to a surgical site and distant from the surgeon, while the proximal end 24 the retractor 10 is the end most proximate the surgeon and distant the surgical site.

Similar to the operation of the opposing arms 14, 16, the hinge 18 is operable to permit movement of one or both of the opposing extensions 28, 30 relative to one another. In this manner, the opposing extensions 28, 30 may be selectively distanced from each other in various configurations so that a void 36 between the opposing extensions 28, 30 is thereby defined. Similar to void 22 of the opposing arms 14, 16, the size of the void 36 is variable and selectively determined by the configuration of the opposing extensions 28, 30.

A locking assembly 38 that is operable to lock the body 12 in a desired configuration is mounted to the grip assembly 26. The locking assembly 38 includes a lever 40, a tab 42, and a spring 44. The lever 40 is secured to the extension 30 and is biased into a locked configuration via the spring 44. The tab 42 is secured to the extension 28 and slidably extends through aperture 46 in the extension 30. The tab 42 has a plurality of notches 48 along an edge 46 of the tab 38 that are each sized and shaped to partially receive a point 50 that projects from the lever 40.

The spring 44 provides a degree of resiliency to the lever 40 so that the point 50 may be selectively pivoted from the locked configuration if the surgeon exerts a force on the lever 40 and automatically returns to the locked configuration if the surgeon releases the lever 40. When in the unlocked configuration, the tab 42 is slidable within the aperture 46 and the opposing extensions 28, 30 and opposing arms 14, 16 may be moved relative to each other. Conversely, when in the locked configuration, the tab 42 is not slidable within the aperture 46 and the opposing extensions 28, 30 and opposing arms 14, 16 may not be moved relative to each other

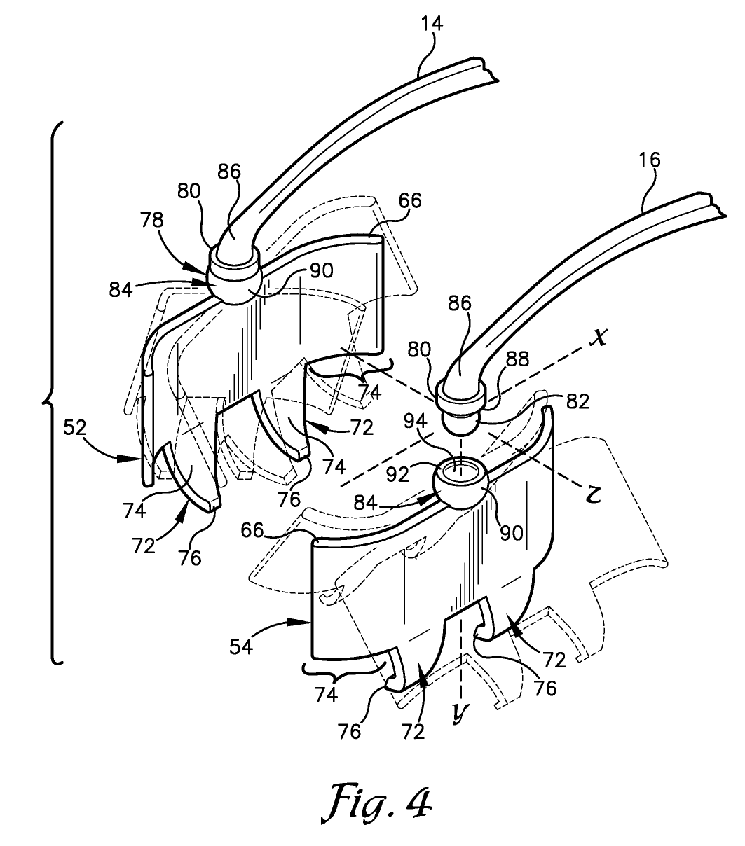

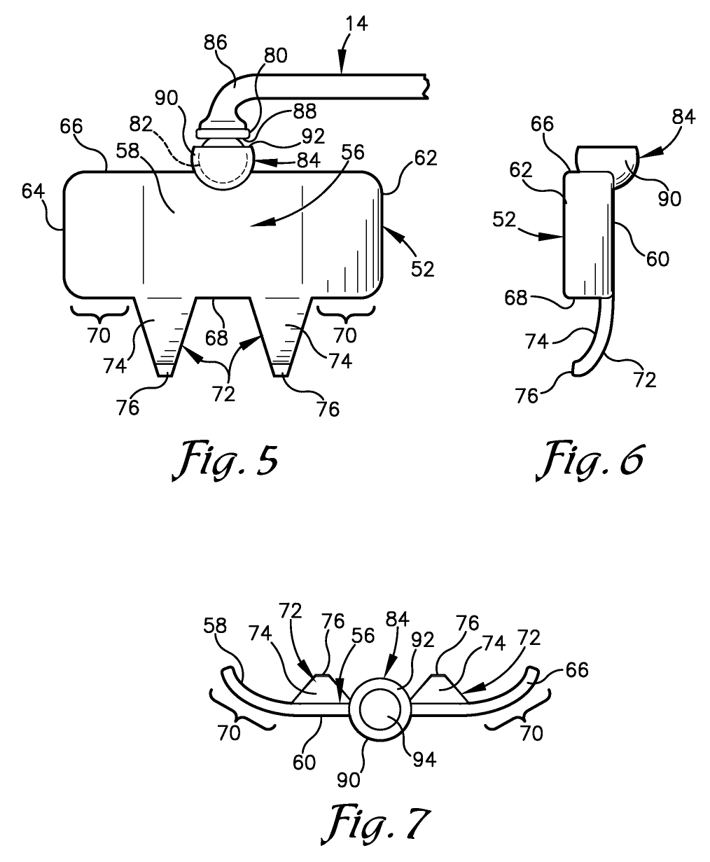

Turning now to FIGS. 4-7, a set of opposing blades 52, 54 are illustrated. The opposing blades 52, 54 are removably attached to each of the opposing arms 14, 16, respectively, at the distal end 18 of the retractor 10. In the exemplary embodiment, the blades 52, 54 are of a uniform thickness and each has parallel front and rear surfaces 58, 60 with a planar face 56 portion in a center thereof. About a perimeter of the front and rear surfaces 58, 60 are parallel side edges 62, 64 that are connected by parallel top and bottom edges 66, 68. Extending from either end of each face 56 of the blades 52, 54 is an edge portion 70 with a degree of curvature relative to the face 56 in a range of 30-60 degrees and preferably 45 degrees from the face 56.

In the exemplary embodiment, the blades 52, 54 are of equal size and shape. It is foreseen, however, that the blades 52, 54 may be of different sizes and/or shapes to enable the retractor 10 to adapt to various applications without deviating from the scope of the present inventive concept. For instance, the degree of curvature of the edge portions 70 may be smaller (e.g., 0 degrees from the face 56), larger (e.g., 90 degrees from the face 56), and/or differ from each other.

Depending from the bottom edge 68 of each of the opposing blades 52, 54 are a plurality of talons 72. Each of the plurality of talons 72 has a curved body 74 having a degree of curvature that is substantially similar to the degree of curvature of the edge portions 70 extending from the face 56. At a distal end of the curved body 74 is a point 76, which is caused to be oriented substantially parallel to the side edges 62, 64 and the front surface 58 via the curved body 74. In the exemplary embodiment, the point 76 is sufficiently dull so as to not damage any part of the patient during use of the present invention.

The curvatures of the edge portions 70 extending from the face 56 and the plurality of talons 72 cooperatively cause the face 56 to be relatively depressed therebetween so that the face 56 is a cavity operable to securely receive a part of the patient therein. Additionally, the curvatures of the edge portions 70 extending from the face 56 are configured to maximize a contact area between the blades 52, 54 and the incision so that slippage of tissue in contact with the rear surface 60 of each of blades 52, 54 is less likely to occur relative to conventional retractors. In this manner, trauma to the tissue is minimized and exposure of the surgical site is maximized.

Each of the blades 52, 54 is removably secured to one of the opposing arms 14, 16 by a coupling assembly 78. In the exemplary embodiment, the coupling assembly 78 is a ball-and-socket assembly 78 that permits various degrees of movement between each of the opposing arms 14, 16 and its respective blade 52, 54. It is foreseen, however, that the coupling assembly 78 could be any like assembly that permits a degree of movement between each of the opposing arms 14, 16 and its respective blade 52, 54.

The ball-and-socket assembly 78 includes a circumferential ring 80, a ball 82, and a socket 84. The ball 82 depends from the circumferential ring 80, which is connected to an elbow extension portion 86 of each of the opposing arms 14, 16. The ring 80 has a circumferential abutment surface 88 about a perimeter of the ring 80 between the ring 80 and the ball 82. The ball 82 is sized and shaped to be removably received within and at least partially housed by the socket 84. The socket 84 is secured to the top edge 66 of each of the opposing blades 52, 54 has a circumferential wall 90 with an abutment ridge 92 and a cavity 94 therein. The circumferential wall 90 of the socket 84 has a degree of resiliency to permit the ball 82 to snap-fit into the socket 84 and to permit the ball 82 to rotate about the socket 84 without becoming disengaged therefrom.

Regarding the snap-fit of the ball 82 and the socket 84, the surgeon may selectively engage or disengage the ball 82 from the socket 84 by applying a degree of force (e.g., by pushing the ball 82 and socket 84 toward each other so that the circumferential abutment surface 88 abuts the abutment ridge 92 at a predetermined angle that causes the ball 82 to separate from the socket 84, or by pulling the ball 82 and socket 84 away from each other). The degree of force necessary to separate the ball 82 from the socket 84 via abutting the circumferential abutment surface 88 and the abutment ridge 92 is less than the degree of force necessary to separate the ball 82 from the socket 84 via pulling them apart. Additionally, the degree of force necessary to engage or disengage the ball 82 from the socket 84 is greater than the force exerted on the ball-and-socket assembly 78 during usage of the retractor 10.

Regarding the rotation of the ball 82 within the socket 84, each of the opposing arms 14, 16 provide its respective blade 52, 54 with a degree of movement independent from its arm 14, 16 along an X axis, a Y axis, and a Z axis, which are defined by its arm 14, 16 via the ball 82. As illustrated in FIG. 4, the X axis runs parallel to the opposing arms 14, 16 and elongated body 12 or horizontally (i.e., side to side), the Y axis runs vertically to the X axis (i.e., up and down), and the Z axis runs horizontally at 90 degrees to the X axis. In this manner, when one of both of the blade 52, 54 are secured to the opposing arms 14, 16, that is, when each ball 82 is engaged to each socket 84, each of the blades 52, 54 are operable to move in one or more of the X, Y, and Z axes independent from the opposing arms 14, 16 and each other.

The range of movement between the ball 82 and the socket 84 along the X and Z axes is limited by the ring 80. In the exemplary embodiment, the circumferential abutment surface 88 about the perimeter of the ring 80 is operable to abut the ridge 92 of the socket 84 when the ball 82 and socket 84 pivots and reaches its limit along the X and/or Z axes. In the exemplary embodiment, the degree of pivot between the ball 82 and the socket 84 along the X and Z axes is in the range of 30 to 60 degrees and preferably 45 degrees. The degree of pivot between the ball 82 and the socket 84 along the Y axis is unlimited, (i.e., the blade 52, 54 may continuously rotate with respect to the arm 14, 16). It is foreseen that the ring 80 may be equipped with an extension or backstop (not illustrated) that depends from the ring 80 and is operable to abut the rear surface 60 of the blade 52, 54 and define a degree of pivot between the ball 82 and the socket 84 along the Y axis of, for example, 270 degrees.

In use, the surgeon makes a surgical incision 96 adjacent to a surgical site 98 (e.g., a broken bone). The surgeon then grips the retractor 10 so that the void 36 between the opposing extensions 28, 30 is maximized, which causes the void 22 between the opposing arms 14, 16 and the blades 52, 54 to be minimized (i.e., the retractor 10 is in the first or stored configuration). If the retractor 10 is not in the compressed configuration, the surgeon may alter the configuration of the retractor 10 by moving the lever 40 of the locking assembly 38 so that the point 50 disengages from the tab 42 and expanding the opposing extensions 28, 30 so that the opposing arms 14, 16 are compressed and are more easily inserted into the incision 96.

The blades 52, 54 are then inserted into the incision 96 so that the rear surface 60 of each of the blades 52, 54 abuts tissue 100 on either side of the incision 96 and the talons 72 abut an internal part 102 (e.g., portions of the bone adjacent to the broken bone) of the surgical site 98 and/or is received within the cavity of the face 56. It is foreseen that the talons 72 may be omitted so that the bottom edge 68, face 56, and/or the side edges 62, 64 abut the internal part 102 without deviating from the scope of the present inventive concept.

Once the retractor 10 has been properly positioned with the talons 72 abutting the internal part 102 or the bottom edge 68, face 56, and/or the side edges 62, 64 abutting the internal part 102, the blades 52, 54 are unable to pivot along the Y axis and secure the tissue 100 away from the surgical site 98. The curved design of the blades advantageously minimizes pressure on the tissue 100 and/or surrounding nerves, vessels, and soft tissues (not illustrated).

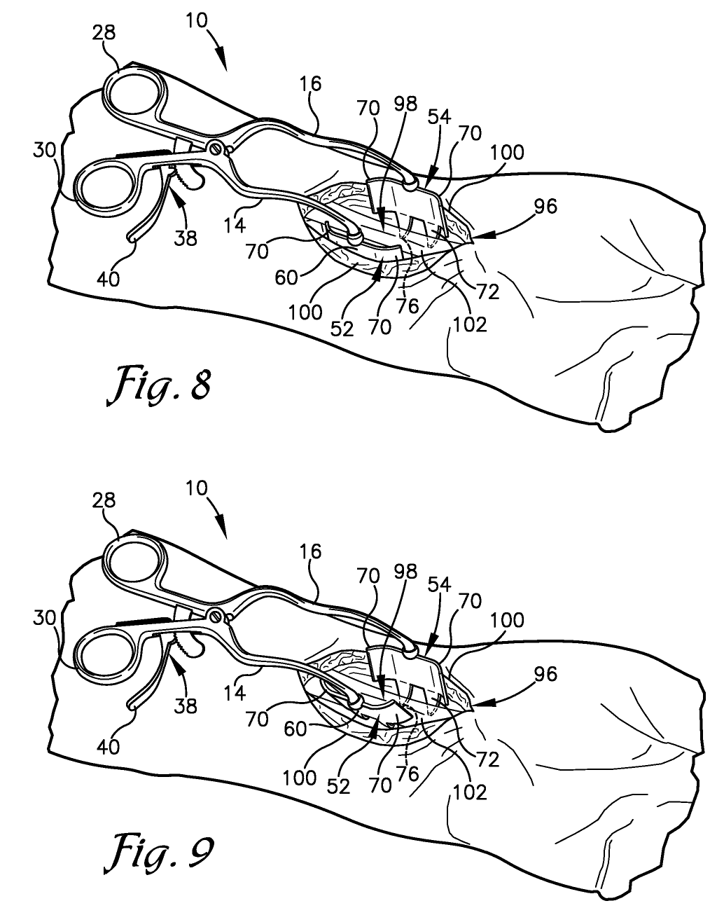

Depending on the application, the surgeon may be required to alter the configuration of the retractor 10 during insertion of the blades 52, 54 and positioning of the talons 72 to permit the internal part 102 of the surgical site 98 to be accommodated in the void of the planar face 56 between the opposing arms 14, 16. Additionally, the blades 52, 54 may be required to pivot along one or more of the X, Y, and Z axes during insertion of the blades 52, 54 to accommodate the size and shape of the internal part 102 so that the curved body 74 of the blades 52, 54 receives, abuts, and partially enwraps the internal part 102, and the points 76 of the talons 72 extend past the internal part 102 and underneath the internal part 102, as illustrated in FIG. 8 so that the internal part 102 is received in or adjacent to the cavity of the face 56. In this manner, a pivot point between the present invention and the internal part 102 is created at or adjacent to the cavity of the face 56 and the blades 52, 54 are slidably and pivotably secured to the surgical site 98 and particularly to the internal part 102 without piercing the internal part 102 or otherwise damaging any other area of the patient.

After the talons 72 are abuttingly secured against and/or slightly beneath the internal part 102 of the surgical site 98, the surgeon unlocks the locking assembly 38 and alters the configuration of the retractor 10 at the pivot point (point of contact between the present invention and the internal part 102) so that the void 36 between the opposing extensions 28, 30 is minimized, which causes the void 22 between the opposing arms 14, 16 and the blades 52, 54 to be maximized (i.e., the retractor 10 is moved to the second or in-use configuration), as illustrated in FIG. 9. At the point, the primary positioning of the retractor 10 is complete. The retractor 10 is also subject to automatic and/or selective secondary positioning as follows.

As the retractor 10 is altered from the stored configuration to the in-use configuration, each of the blades 52, 54 pivots independently with respect to the opposing arms 14, 16 along the X, Y and Z axes. The blades 52, 54 are not anchored to the internal part 102 and are operable to automatically and slidable move relative to the internal part 102, if necessary, to minimizes stress on the tissue 100 of the incision 96. Additionally, as the retractor 10 moves from the stored configuration to the in-use configuration, the surgeon may selectively move the blades 52, 54 of the retractor 10 further underneath the internal part 102 to further secure the retractor 10 to the internal part 102 and/or obtain a better perspective of the surgical site 98. This automatic and/or selective secondary movement of the blades 52, 54 advantageously provides better leverage and a wider opening than what would have been provided without the secondary positioning of the retractor. When the retractor 10 is in the in-use configuration, the contact area between the rear surface 60 and edge portions 70 of each of the blades 52, 54, and the tissue 100 on either side of the incision 96 is maximized so that the likelihood of any slippage of the tissue 100 is decreased relative to conventional retractors. The retractor 10 is then locked in the in-use configuration via the locking assembly 38. It should also be noted that the retractor 10, enables the surgeon to make slight adjustments, as desired, during the course of a surgical procedure via the secondary movement.

Accordingly, the retractor 10 is operable to open the surgical site 98 to a maximum while maximizing contact area between the tissue 100 and the blades 52, 54 and leveraging on the internal part 102 of the surgical site 98 so that slippage of the retained tissue 100 with respect to the blades 52, 54 and inadvertent movement of the retractor 10 with respect to the surgical site 98 is prevented, and repositioning of the retractor 10 is less likely to be required by the surgeon. Thus, the present inventive concept ensures that the time required to perform a surgical procedure with the retractor 10 is kept to a minimum relative to convention retractors, which causes trauma, recovery time, risk, and costs of the surgical procedure to be minimized relative to convention retractors.

Having now described the features, discoveries and principles of the general inventive concept, the manner in which the general inventive concept is constructed and used, the characteristics of the construction, and advantageous, new and useful results obtained; the new and useful structures, devices, elements, arrangements, parts and combinations, are set forth in the appended claims.

It is also to be understood that the following claims are intended to cover all of the generic and specific features of the general inventive concept herein described, and all statements of the scope of the general inventive concept which, as a matter of language, might be said to fall therebetween.

What is claimed is:

1. A self-retaining retractor assembly comprising:
an elongated body having opposing arms that are hinged together at a connection point that is spaced from either end of the elongated body; and
a spacer coupled to and depending from each of the arms, each of the spacers having a plurality of axes and operable to independently pivot with respect to the elongated body about each of the plurality of axes, the spacer operable to partially enwrap a portion of a surgical site.

2. The self-retaining retractor assembly according to claim 1, wherein the plurality of axes include an X axis, a Y axis, and a Z axis.

3. The self-retaining retractor assembly according to claim 1, further comprising:
a coupling assembly operable to permit independent removal of one or both spacers from the arms.

4. The self-retaining retractor assembly according to claim 3, wherein the coupling assembly is a ball-and-socket coupling assembly having a ball depending from each of the arms that is operable to be received via a snap-fit into an aperture in each of the spacers, each of the apertures defined by a circumferential wall having a degree of resiliency.

5. The self-retaining retractor assembly according to claim 3, wherein the coupling assembly provides each of the spacer with a first degree of pivot about the X and Z axes, and a second degree of pivot about the Y axis.

6. The self-retaining retractor assembly according to claim 5, wherein the first degree of pivot is defined by an annular ring on each of the arms, the annular rings limiting the first degree of pivot of each of the spacers to about 45 degrees.

7. The self-retaining retractor assembly according to claim 5, wherein the second degree of pivot is perpetual.

8. The self-retaining retractor assembly according to claim 3, wherein the plurality of axes of each the spacers intersect at the coupling assembly of each of the spacers.

9. The self-retaining retractor assembly according to claim 1, wherein each of the spacers include a face surface with side edge portions and a bottom edge portion, the bottom edge portion having at least one talon depending therefrom, the side edge portion and the at least one talon being concave to form a cavity on each of the face surfaces.

10. The self-retaining retractor assembly according to claim 1, wherein the spacer includes at least one talon having a curved surface operable to partially enwrap the portion of the surgical site.

11. The self-retaining retractor assembly according to claim 1, wherein the spacer includes two talons each having a curved abutment surface operable to cooperatively pivot and align the spacer with respect to the elongated body when the curved surfaces slidably abut the portion of the surgical site.

12. The self-retaining retractor assembly according to claim 11, further comprising:
a locking mechanism on the body that is operable to lock the arms in one of a plurality of orientations with respect to each other; and
a spring on the locking mechanism that is operable to bias the locking mechanism to a locked configuration.

13. The self-retaining retractor assembly according to claim 1, wherein the spacers are radiolucent blades.

14. The self-retaining retractor assembly according to claim 1, wherein the connection point is a hinge that is operable to permit movement of at least one of the arms with respect to the other of the arms.

15. A surgical retractor comprising:
a plurality of arms that are hinged together; and
a blade coupled to each of the arms via a friction-fit engagement; and
a divider between each of the plurality of arms and each of the blades that is operable to (i) define a limited range of movement the blade and the arm, and (ii) separate the blade from the arm if the blade exceeds the limited range of movement.

16. The surgical retractor according to claim 15, wherein the blade is rotatable relative to the arm.

17. The surgical retractor according to claim 15, wherein the limited range of movement provided to each of the blades includes a plurality of axes, the plurality of axes including an X axis, a Y axis, and a Z axis, the plurality of axes having an intersection point on the blade.

18. A method of surgery comprising the steps of:
providing a retractor having a plurality of opposing blades depending from hinged arms that are operable to selectively define a void between the plurality of blades;
setting the hinged arms in a first configuration with respect to each other, the first configuration defining a first plurality of axes of each of the plurality of blades and permitting independent movement of the plurality of blades along the first plurality of axes;
inserting the plurality of blades at least partially into a surgical area with the hinged arms in the first configuration;
partially enwrapping a portion of the surgical site via an abutment portion of the spacer so that the portion of the surgical site is slidably received by the abutment portion of the spacer; and
retracting tissue adjacent to the surgical area by moving the hinged arms from the first configuration to a second configuration, the second configuration defining a second plurality of axes of each of the plurality of blades and permitting independent movement of the plurality of blades along the second plurality of axes.

19. The method of surgery according to claim 18, wherein the step of partially enwrapping the portion of the surgical site includes pivoting the spacer with respect to the elongated body when another abutment portion of the spacer and the abutment portion of the spacer partially enwrap the portion of the surgical site.

20. The method of surgery according to claim 18, wherein the step of retracting tissue adjacent to the surgical area includes levering the abutment portion of the spacer on the portion of the surgical area as the hinged arms are moved from the first configuration to the second configuration.
